(19) United States
(12) Patent Application Publication
(10) Pub. No.: US 2021/0324455 A1
(43) Pub. Date: Oct. 21, 2021

(54) In-Vitro Method and Device for Detecting a Target Nucleic Acid

(71) Applicant: ICHORtec GmbH, Roetgen (DE)

(72) Inventor: Oleh FEDORYCH, Aachen (DE)

(21) Appl. No.: 16/860,765

(22) Filed: Apr. 28, 2020

(30) Foreign Application Priority Data
Apr. 16, 2020 (EP) 20169908.9

(51) Int. Cl. C12Q 1/6825; B01L 7/00; C12Q 1/6806; G01N 1/10; G02B 21/16; G02B 21/06; G02B 21/18

(52) U.S. Cl. CPC C12Q 1/6825; B01L 7/52; C12Q 1/6806; G01N 1/10; B01L 2300/0663; G02B 21/06; G02B 21/18; B01L 2200/10; G02B 21/16

(57) ABSTRACT

Disclosed herein is an in-vitro method for detecting a target nucleic acid in a patient's sample. An exemplary method involves steps of bringing the sample in contact with a probe, that is a molecular beacon, is labeled with a fluorescent dye, and can hybridize with the target, then irradiating the probe with light exciting the fluorescent dye, then observing the sample with a detector, and then, if the detector detects light emitted by the probe, concluding that the target is in the sample. In order to rapidly detect the emitted light, prior to bringing the probe in contact with the sample, the probe can be immobilized in a carrier element, wherein the carrier element is in contact with an optical element which lets the emitted light pass to the detector.

### CROSS-REFERENCE TO RELATED APPLICATIONS

This application claims priority to and benefit of European Patent Application No. 20169908.9 filed on 16 Apr. 2020, which is incorporated by reference in its entirety.

### REFERENCE TO SEQUENCE LISTING

The Sequence Listing submitted Apr. 28, 2020, as a text file named “066507_001_seqlist.txt” created on Apr. 28, 2020, and having a size of 463 bytes is hereby incorporated by reference pursuant to 37 C.F.R. § 1.52(e)(5).

### TECHNICAL FIELD OF THE INVENTION

This invention is generally related to devices and methods of their use for detecting nucleic acids in a sample.

### BACKGROUND OF THE INVENTION

Viral and bacterial infections of the human body represent a major global public health concern in the 21st century. Appropriate treatment depends on a correct diagnosis, not only of the type of the infectious agent, but also of the load or degree of infection. Presently most diagnostic methods for detecting infections are based on the detection of the DNA of the infectious agent by polymerase chain reaction (PCR) techniques which usually take about 90 minutes to 3 hours. PCR allows to make a large number of copies of a nucleic acid sample, thus allowing scientists to take a very small sample and amplify it to a large enough amount to study it in detail. PCR is a powerful tool widely used in in-vitro diagnostics. The method allows detecting the presence of individual RNA/DNA in a patient's sample which makes PCR more reliable in comparison with immunoassay (ELISA) or similar techniques. Several modifications of the PCR method were also developed, e.g. reverse transcription PCR (RT-PCR), real time PCR or quantitative PCR (qPCR). However, a reliable detection of target nucleic acids within few minutes and outside of the laboratory is currently not possible with any available technique.

Thus, the object of the present invention is to develop a device and a method for detecting target nucleic acids quickly and reliably. Since the detection is based on fluorescence measurement, the invention shall speed up detection of the emitted light.

### SUMMARY OF THE INVENTION

Disclosed herein are in-vitro methods for detecting a target nucleic acid in a sample. An exemplary method involves steps of bringing the sample in contact with a probe, that is a molecular beacon, is labeled with a fluorescent dye, and can hybridize with the target, then irradiating the probe with light exciting the fluorescent dye, then observing the sample with a detector, and then, if the detector detects light emitted by the probe, concluding that the target is in the sample. Prior to contacting the probe with the sample, the probe can be immobilized in a carrier element, wherein the carrier element is in contact with an optical element which lets the emitted light pass to the detector. The carrier element can be a polymer. In one embodiment, the carrier element contains at least 20 nmol/1 of the probe, preferably at least 2.5 μmol/1 of the probe. In one embodiment, the excitation light is a laser beam.

In another embodiment, prior to performing step (b) the carrier element is heated to at least 65° C., the carrier element is exposed to ultraviolet radiation, or a combination of both.

In one embodiment, a peak wavelength of the emitted light that is specific to a hybrid of the target and the probe is defined, and if the emitted light has the peak wavelength, it is concluded that the target is in the sample.

Also disclosed is an analyzing device for performing the disclosed methods, the devices including a light source (12, 22, 31) for the excitation light, and a microprocessor that is arranged for automatically receiving data from the detector (15, 29, 39), and for concluding that the target is in the sample. The analyzing device further includes a beam splitter with high pass optical filter, or a dichroic mirror between the carrier element (7, 26, 35) and the detector (15, 29, 39) which eliminates the excitation light from a signal to be measured by the detector (15, 29, 39). The optical element (8, 25, 36) can be an optical fiber, or a glass window. The analyzing device can include at least one focusing element (18, 24, 34) between the light source (12, 22, 32) and the carrier element (7, 26, 35) which delivers the excitation light onto the carrier element (7, 26, 35). The analyzing device can include at least one beam collimating element (18, 24, 34) between the carrier element (7, 26, 35) and the optical element (8, 25, 36) which collects the emitted light to the optical element (8, 25, 36).

In another embodiment, a sampling unit (2) is disclosed for adding into an analyzing unit (3, 21, 31) whereby the sampling unit (2) and the analyzing unit (3, 21, 31) form an analyzing device (1, 30) according to one of claims 8 to 12, characterized in that the sampling unit (2) contains the carrier element (7, 26, 35) and the optical element (8, 25, 36), and a container for collecting the patient's sample, and for isolating the sample from the environment, wherein the container in particular is a pipette.

### BRIEF DESCRIPTION OF THE DRAWINGS

FIG. 1A is a first analyzing device according to the invention. FIG. 1B is the analyzing unit of the first device of FIG. 1A.

FIG. 2 is an analyzing unit of another device according to the invention.

FIG. 3 is a second analyzing device according to the invention.

FIG. 4 shows the fluorescence spectra of samples with and without target RNA.

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “nucleic acid” or “polynucleic acid” refers to the order or sequence of nucleotides along a strand of nucleic acids. The nucleic acid sequence may be single-stranded or double-stranded or contain portions of both double-stranded and single-stranded sequences. The nucleic acid sequence may be composed of DNA, both genomic and cDNA, RNA or DNA/RNA hybrid.

As used herein, “molecular beacons”, or “(molecular beacon) probes”, are oligonucleotide hybridization probes that can report the presence of specific nucleic acids in homogenous solutions. Molecular beacons are hairpin-shaped molecules with an internally quenched fluorescent dye whose fluorescence is restored when they bind to a target nucleic acid sequence. This is a novel non-radioactive method for detecting specific sequences of nucleic acids. They are useful in situations where it is either not possible or desirable to isolate the probe-target hybrids from an excess of the hybridization probes.

A typical molecular beacon probe is 25-40 nucleotides long. The middle nucleotides are complementary to the target DNA or RNA and do not base pair with one another, while the five to seven nucleotides at each terminus are complementary to each other rather than to the target DNA. A typical molecular beacon structure can be divided in 4 parts: 1) loop, an 18-30 base pair region of the molecular beacon that is complementary to the target sequence; 2) stem formed by the attachment to both termini of the loop of two short (5 to 7 nucleotide residues) oligonucleotides that are complementary to each other; 3) 5′ fluorescent dye at the 5′ end of the molecular beacon, a fluorescent dye is covalently attached; 4) 3′ quencher (non-fluorescent) dye that is covalently attached to the 3′ end of the molecular beacon. When the beacon is in closed loop shape, the quencher resides in proximity to the fluorescent dye, which results in quenching the fluorescent emission of the latter. If the nucleic acid to be detected is complementary to the strand in the loop, the event of hybridization occurs. The duplex formed between the nucleic acid and the loop is more stable than that of the stem because the former duplex involves more base pairs. This causes the separation of the stem and hence of the fluorescent dye and the quencher. Once the fluorescent dye is no longer next to the quencher, illumination of the hybrid with light results in the fluorescent emission. The presence of the emission reports that the event of hybridization has occurred and hence the target nucleic acid sequence is present in the test sample. The fluorescent dye of the molecular beacon may be any suitable fluorescent dye, for example ABI dyes (e.g. FAMTM, HEXTM, TETTM, JOETM, ROXTM, CAL FluorTM Red 610), cyanine dyes (e.g. Yakima Yellow or ATTO) or molecular dyes (e.g. ALEXA-fluor or BODIPY dyes). The quencher of the molecular beacon may be any suitable quencher, for example TAM, BHQ1, BHQ2, DAB, Eclip, BBQ650. Molecular beacons are synthetic oligonucleotides whose preparation is well documented in the literature (Tyagi S; Kramer FR (1996). “Molecular beacons: probes that fluoresce upon hybridization”. Nat. Biotechnol. 14 (3): 303-308). Further, the molecular beacon probe design is within the skill of a molecular biologist and there are many bioinformatics tools for this purpose available (e.g. Beacon Designer™). In addition, molecular beacon probes are commercially available for many target sequences (e.g. Eurofins Genomics, Ebersberg, Germany; Integrated DNA Technologies, Inc., Coralville, Iowa, USA.

In the context of this invention, “hybridization” means hydrogen bonding, which may be Watson-Crick, Hoogsteen or reversed Hoogsteen hydrogen bonding, between complementary nucleoside or nucleotide bases. Hybridization takes place under stringent or non-stringent conditions. As used herein, “stringent” refers to the conditions, i.e. temperature, buffer composition or ionic strength under which hybridization between polynucleotides occurs. These conditions depend mainly on the composition and complexity of the target nucleic acid and length of the probe. For the hybridization temperature conditions the “Tₘ” (melting temperature) of the nucleic acids has to be considered. “Tₘ” means under specified conditions the temperature at which half of the nucleic acid sequences are disassociated and half are associated. Generally, suitable hybridization conditions may be easily determined by a person skilled in the art.

“Complementary” as used herein, refers to the capacity for pairing between two nucleotides. For example, if a nucleotide at a certain position of an oligonucleotide is capable of hydrogen bonding with a nucleotide at the same position of a DNA or RNA molecule, then the oligonucleotide and the target DNA or RNA are considered to be complementary to each other at that position. For example, the sequence 5′-A-C-T-3′ is complementary to the sequence 3′-T-G-A-5′.

Complementarity may be partial, in which only some of the nucleotides match according to base pairing, or complete, where all the nucleotides match according to base pairing. For purposes of the present invention “substantially complementary” refers to 90% or greater identity over the length of the target base pair region. The complementarity can also be 45, 50, 60, 70, 75, 80, 85, 90, 91, 92, 93, 94, 95, 96, 97, 98, 99, or 100% complementary, or any amount below or in between these amounts. In other words, the oligonucleotide and the target DNA or RNA are complementary to each other when a sufficient number of corresponding positions in each molecule are occupied by nucleotides which can build a hydrogen bond with each other. Thus, “specifically hybridizable” and “complementary” are terms which are used to indicate a sufficient degree of complementarity or precise pairing such that stable and specific binding occurs between the probe and the DNA or RNA target. It is understood in the art that the probe does not need to be 100% complementary to that of its target nucleic acid to be specifically hybridizable.

“Oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly.

“Subject” or “patient” as used herein refers to a mammalian individual, including murines, cattle, simians and humans. Preferably, the patient is a human.

“Sample” as used herein refers to a biological sample encompassing liquid and solid samples. Liquid samples encompass saliva, sputum, sweat, urine, nasal secretion, bronchoalveolar lavage fluid, laryngo-pharyngeal scrape test, vaginal secretion, blood liquids (e.g. serum, plasma) and cerebrospinal fluid (CSF). Solid samples encompass tissue samples such as tissue cultures or biopsy specimen. A preferred patient's sample is sputum. The patient's sample will be collected with the disposable pipette as described below.

### II. In-Vitro Method And Device For Detecting A Target Nucleic Acid

Proper diagnosis and treatment of viral and bacterial infections rely on accurate detection of the microbes within the subject. Presently most diagnostic methods for detecting infections are based on the detection of the DNA of the infectious agent by polymerase chain reaction (PCR) techniques which usually take about 90 minutes to 3 hours. Disclosed herein are devices and methods for rapidly and accurately detecting nucleic acids in a sample.

### A. In-Vitro Method For Detecting Nucleic Acid

An exemplary method involves steps of (a) bringing the sample in contact with a probe, that is a molecular beacon, is labeled with a fluorescent dye, and can hybridize with the target, then (b) irradiating the probe with light exciting the fluorescent dye, then (c) observing the sample with a detector, and then, (d) if the detector detects light emitted by the probe, concluding that the target is in the sample. Prior to contacting the probe with the sample, the probe can be immobilized in a carrier element, wherein the carrier element is in contact with an optical element which lets the emitted light pass to the detector.

Without being bound by any particular theory, it is believed that the emitted light of a low number of target-probe hybrid molecules, or even of one single such molecule can be detected when in the focus of the detector. The method avoids false positive emission of non-hybridized probe molecules. The method mechanically fixes the hybridized molecules in the focus of the detector and thus enables detection of each single hybrid, without amplification (e.g. PCR or any other thermal amplification using polymerase).

As mentioned above, step (a) of the method of the present invention is based on labeling a nucleic acid contained in a patient's sample (i.e the target nucleic acid) with a probe. According to the present invention this probe is a molecular beacon. This probe (i.e. molecular beacon) is immobilized to a carrier element and hybridizes to the target nucleic acid under appropriate conditions. It is not necessary to extract the target nucleic acid (RNA or DNA) in advance since the method is very sensitive and is able to detect less than 10 copies. However, depending on the sample obtained, extraction of the target nucleic acid by generally known methods and/or commercially available extraction kits may be made in advance. In addition, when a double-stranded nucleic acid (DNA or RNA) has to be detected a strand separation has to be made in advance. This strand separation may be conducted e.g. chemically, with UV-light or by heat treatment.

In one embodiment, the carrier element is preferably a polymer, in particular a liquid swelling polymer. Examples are agarose gel, hydrogel, polyamide gel or polyester gel. In this method, the polymer has several functions: (1) binding a large number of probe molecules, (2) absorbing target molecules from the sample, (3) purification of the target molecules and (4) immobilizing the probes around an optical fiber so that the fluorescence appearing in result of hybridization has larger probability of coupling to the optical fiber.

Agarose is well known in molecular biology laboratory application, and in particular easy to adapt for binding and absorbing specific molecules.

In another embodiment, the carrier element preferably contains at least 20 nmol/1, preferably at least 2.5 μmol/1 of the probe. In this method, almost every target molecule migrating into the carrier element is immediately in contact with a probe molecule that is ready for hybridizing.

In one embodiment of the present invention, the method is used for detecting a virus in a patient's sample. “Virus” or “viral” means any single- or double-stranded DNA or RNA virus. In particular, Human Immunodeficiency Virus (HIV), Zika, MERS-Corona, SARS-CoV-1, Sars-CoV-2, Hepatitis B Virus (HBV), Hepatitis C Virus (HCV), Human Lyphotropic Virus-type 1-4 (HTLV-1-4), Epstein-Barr Virus (EBV), Human Papilloma Virus (HPV), Influenza A, B or C, Non-Influenza Respiratory Viruses (NIRVs, e.g. respiratory syncytial virus, parainfluenza virus, rhinovirus, metapneumovirus), norovirus, Ebola virus, Reovirus, Herpes virus (simplex 1, 2+B, 5, 6, 7, 8), Pox virus, FSME virus, Dengue virus, Rubivirus, Varizella zoster virus and Cytomegalievirus.

In one embodiment, the target nucleic acid may in particular be a virus RNA. This method provides for screening large numbers of patients for infection, in an extremely reduced time scale, compared to the state of the art. The virus may in particular be a coronavirus, further in particular SARS-CoV-2. The method is thus applicable in a recent world-wide pandemic situation.

In another embodiment, the excitation light may in particular be a laser beam. This method allows for limiting the excitation light to the excitation wavelength of the fluorescent dye, and thus for avoiding side effects induced by other wavelengths.

In one embodiment, before irradiating the carrier with the excitation light, the carrier is heated to at least 65° C. Additionally, or alternatively, before irradiating the carrier with the excitation light, the carrier is exposed to ultraviolet radiation. Both heating and exposing to UV radiation removes the capsid around the target nucleic acid and increases the release of the target nucleic acid from the sample. In case of a double-stranded nucleic acid it may also lead to denaturation.

In another embodiment, a peak wavelength of the emitted light is defined that is specific to a hybrid of the target and the probe, and if the emitted light has the peak wavelength, it is concluded that the target is in the sample. This method is based on the finding that each target-probe hybrid has a unique wavelength of emitted light, and allows for identifying any known target, in a patient's sample. In particular, the peak wavelength λₑₘ may be calculated from h·c/λₑₘ=h·c/λ_{ref}−E₀, wherein ref is a reference emission of the fluorescent dye without hybridization, h is the Planck constant, c is the speed of light and E₀ is the hybridization energy of the target-probe hybrid, as known from Santalucia J Jr, Proc. Natl. Acad. Sci. USA 95 (1998) and calculated in terms of Nearest-Neighbor (NN) model, where change of the energy is 0.0243 eV.

### B. Analyzing Device

The present invention further concerns an analyzing device for performing the disclosed methods, having a light source for the excitation light, and a microprocessor that is arranged for automatically receiving data from the detector, and for concluding that the target is in the sample. This device allows for automatic execution of the disclosed method.

In one embodiment, the device has a beam splitter with high pass optical filter, or a dichroic mirror between the carrier element and the detector which eliminates the excitation light from a signal to be measured by the detector. In this device, only the emitted light is protruding to the detector.

In another embodiment, the optical element is an optical fiber. In an alternative device according to the invention, the optical element is a glass window. Optical fibers and glass windows are commonly known and readily available for building a device according to the invention.

In one embodiment, the device has at least one focusing element between the light source and the carrier element which delivers the excitation light onto the carrier element. In this device, the excitation effect onto the probe is improved.

In another embodiment, the device has at least one beam collimating element between the carrier element and the optical element which collects the emitted light to the optical element. In this device, detection of the emitted light is improved.

The present invention further concerns a sampling unit for adding into an analyzing unit whereby the sampling unit and the analyzing unit form such analyzing device, the sampling unit having the carrier element and the optical element, and a container for collecting the patient's sample, and for isolating the sample from the environment, wherein the container in particular is a pipette. This unit allows for secure handling of infectious matter. The sampling unit may be a low-cost one-time disposable product.

### C. Exemplary Devices

Preferred embodiments of the invention are described with reference to the figures, showing:

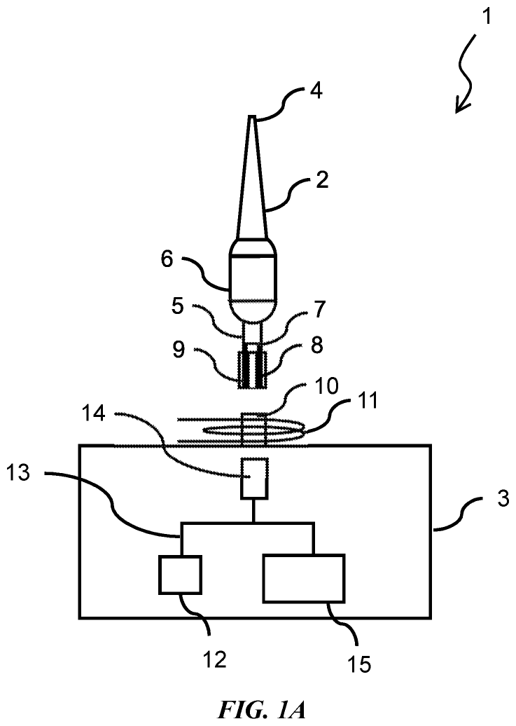

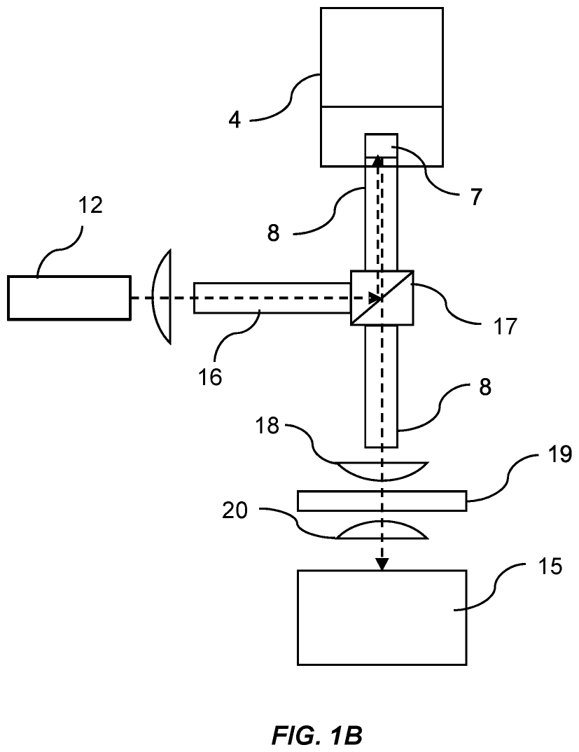

in FIG. 1A an first analyzing device according to the invention,

in FIG. 1B the analyzing unit of the first device,

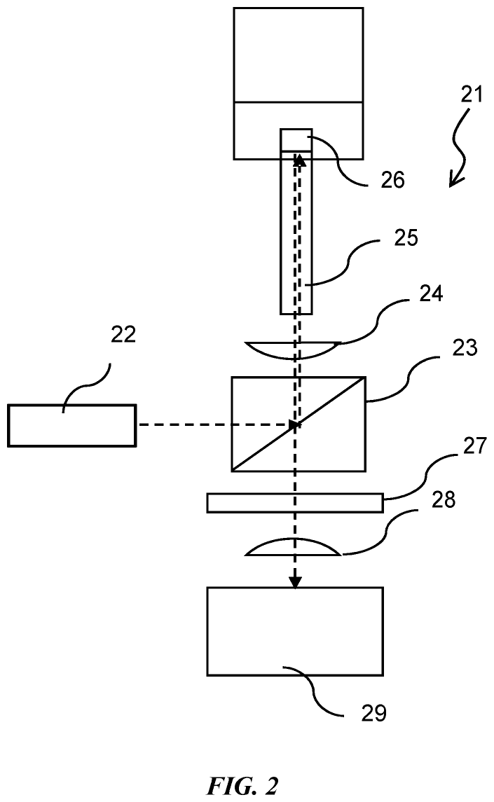

in FIG. 2 an analyzing unit of another device according to the invention,

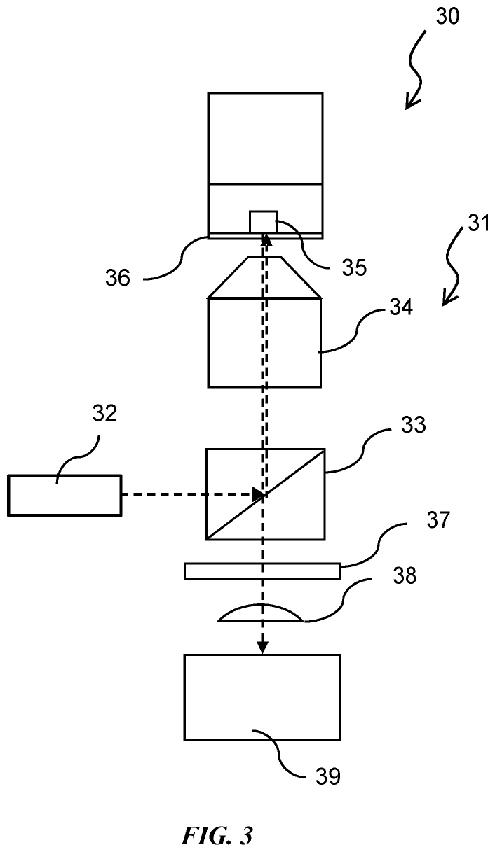

in FIG. 3 a second analyzing device according to the invention, and

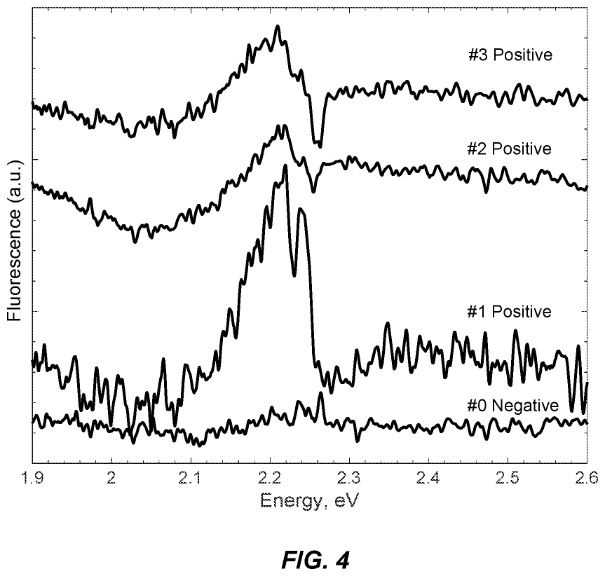

in FIG. 4. fluorescence spectra of samples with and without target RNA The first analyzing device 1 shown in FIG. 1A describes a disposable sampling unit 2 and an analyzing unit 3, which is in detail shown in FIG. 1B.

The sampling unit 2 is formed as a pipette, and comprises an extraction nozzle 4, a reaction chamber 5 surrounded by flexible walls 6, a carrier element 7 inside the reaction chamber 5, an optical element 8 commutating the carrier element 7 with the analyzing unit 3, and a ferrule 9 for aligning the optical element 8 to the same.

The extraction nozzle 4 has a single use liquid tide cap (not shown) for locking the nozzle 4 after extraction of the patient's sample (not shown) by pressing and releasing the walls 6. The carrier element 7 is a block of 20 nl of polymerized agarose, which carries 2.5 μmol/1 of beacon probe molecules labeled with a fluorescent dye, and designed to hybridize with SARS-CoV-2 RNA.

To provide sterility in delivery, the sampling unit 2 is packed individually in clean plastic (not shown). In another analyzing device according to the invention, the sampling unit 2 is packed in a similar bag or container. The sampling unit 2 has respective marks and labels that the control unit may recognize what kind of test to perform. In another analyzing device according to the invention, the sampling unit 2 has an RFID chip. The analyzing unit 3 also can send request into a data base in order to validate the sampling unit 2 (e.g. expiration date, was the tester used before).

The analyzing unit 3 comprises an insertion slot 10 for the sampling unit 2 with a heating element 11 for speeding-up of the hybridization, a light source 12 commutated with a Y-fiber 13 and an optical fiber connector 14. The connector 14 serves for efficient transfer of light power from the light source 12 to the optical element 8, and to the carrier element 7. The second arm of the Y-fiber 13 redirects fluorescence from the carrier element 7 to a detector 15, namely a spectrometer.

In other embodiments of the invention, the analyzing unit 3 is equipped with a system enabling detection when new pipette inserted into the slot 10, and/or with a computer or other device capable to readout spectra from detector 15 and perform inline mathematical analysis of measured spectra, and/or other optical elements for improving quality of the RNA/DNA detection, and/or an automatic shutter to protect the slot 10 from contamination, and/or an additional light source 12 for detecting presence of a sample, in the container.

In order to eliminate dust contamination of the open end of the optical element 8, a protection cup is used. The open side of the sampling unit 2 is protected with a cup after collection of the patient's sample (both cups not shown).

For collecting a sample, an operator of the analyzing device 1 (the patient him- or herself or health personnel) inserts the sampling unit 2 into the patient's mouth, presses with fingers on the walls 6 of the reaction chamber 5 and releases until few drops of sputum are sucked in, and seals the nozzle 4 with the respective cup. For detecting SARS-CoV-2 in the patient's sample, the operator removes the protection cup from the optical element 8, and inserts the sampling unit 2 into the slot 10, and initiates analysis procedure by pressing a start button (not shown) on the analyzing unit b. After obtaining the result, the operator removes the sampling unit 2 from the slot 10 and puts it into a gated bin for following disintegration/disinfection of the sampling unit 2 and its content.

The light source 12 emits laser light with 532 nm. The light source 12 emits laser light into an excitation arm 16 of the Y-fiber 13. A beam splitter 17 reflects 10% of the laser power to the carrier element 7 through the optical element 8. Light emitted by the fluorescent dye is collected by the optical element 8. After the beam splitter 17, 90% of the emitted light is collected by a focusing and beam collimating element 18, namely a lens. Scattered laser light is filtered from the emitted light by a high pass filter element 19, and collected on an entrance slit of the detector 15 by a further focusing lens 20.

FIG. 2 shows a second analyzing unit 21 of yet another analyzing device according to the invention (not shown). In the analyzing unit 21, 10% of laser light emitted from a light source 22 similar to the first analyzing unit 3 is reflected by a beam splitter 23 to a focusing and beam collimating element 24, namely a lens. In another analyzing device according to the invention, the focusing and beam collimating element 24 is an optical fiber collimator. An optical element 25 delivers the light to the carrier element 26. Light emitted by the fluorescent dye (at least part of it) is collected with the optical element 25 and delivered to the focusing and beam collimating element 24. 90% of the emitted light passes through the beam splitter 23 and further through a high pass optical filter element 27. A further lens 28 focuses the emitted light to a detector 29. In another configuration the beam splitter 23 and the optical fiber 27 can be replaced with respective dichroic mirror, which has to reflect the wavelength emitted by the laser and transmit fluorescent light.

FIG. 3 shows a second analyzing device 30 according to the invention. In an analyzing unit 31 of the second analyzing device 30, 10% of laser light emitted from a light source 31 again similar to the first analyzing unit 3 is reflected by a beam splitter 33 towards a focusing and beam collimating element 34, namely a planar micro objective. The focusing and beam collimating element 34 focuses excitation light on a carrier element 35 through an optical element 36, namely a glass window. Light emitted by the fluorescent dye passes through the optical element 36 and is collected by the micro-objective. 90% of the emitted light passes through the beam splitter 33 and further through a high pass optical filter element 37. A further lens 38 focuses the emitted light to a detector 39.

### EXAMPLES

### Example 1: Micro-Fluorescence Measurement

### Materials and Methods:

Agarose powder was weighed and mixed with a buffer solution (e.g. 0.1 M phosphate buffer, pH 6.5 or 20 mM Tris-HCl buffer, pH8.0), so that a 4.0% agarose gel was obtained after heating of the mixture. The mixture was heated up to 90° C. and carefully mixed for about 10 min. 10 μL of the gel was retrieved with a pipette and inserted into a cuvette. The cuvette with agarose gel was kept at 60-70° C.

10 μL of a solution 20 mM Tris-HCl buffer, pH8.0, containing 2.5 μM/1 of Corona E-Beacons labeled with Rhodamine 6G and quenched with BMN-Q620 (biomers.net GmbH Ulm, Germany) was retrieved and inserted into the cuvette containing the 10 μL of agarose gel.

The Corona E-Beacon had the sequence:

(SEQ ID NO: 1)
Rhodamine 6G-ACACTAGCCATCCTTACTGCGCTTCG-BMNQ620

This results in a final concentration of around 2% agarose. The mixture was mixed for about 30 seconds and was deposited with help of another pipette on the bare end of an optical fiber. The resulting polymer layer had a volume of much less than 20 nL.

Four test samples were analyzed:
1. Extracted RNA (in 20 mM Tris-HCl buffer, pH 8.0) obtained from a human patient and previously analyzed (passed through) with PCR. The test sample is characterized by absence of the virus RNA—#0 Negative
2. Extracted RNA (in 20 mM Tris-HCl buffer, pH 8.0) obtained from a human patient and previously analyzed (passed through) with PCR. The test sample had low concentration (about 100 targets per reaction) of the virus RNA—#1 Positive
3. Extracted RNA (in 20 mM Tris-HCl buffer, pH 8.0) obtained from a human patient and previously analyzed (passed through) with PCR. The test sample had higher concentration (about 3,000) of the virus RNA—#2 Positive
4. Extracted RNA (in 20 mM Tris-HCl buffer, pH 8.0) obtained from a human patient and previously analyzed (passed through) with PCR. The test sample had very high concentration (about 3,700,000) of the virus RNA—#3 Positive

The optical fiber with the polymer layer was immersed into the “#0 Negative” test sample. The fluorescence spectra of the “#0 Negative” sample were measured for 4-5 min.

Another optical fiber with the polymer layer with exactly the same concentration of molecular beacons was one after another immersed into #1 Positive, #2 Positive and #3 Positive and the fluorescence spectra were each collected for about 4-5 min.

Results:

The result of the fluorescence measurement is shown in FIG. 4. As can be seen from FIG. 4, the fluorescence measured in the Positive samples is at 2.208 eV. In accordance with the molecular beacons as probes specification they were labeled with Rhodamine 6G with maximum emission at 2.232 eV. The difference between expected and observed fluorescence is 0,0243 eV could be attributed to lowering of energy due to primer—target hybridization, so E1>E2. In terms of temperature, probe—target hybridization leads to lowering of the free energy of RNA-Molecular Beacon hybrid (ΔG)=−0.558 kcal/mol.

In the figures are the following:

1. Analyzing device

2. Sampling unit

3. Analyzing unit

4. Extraction nozzle

5. Reaction chamber

6. Flexible wall

7. Carrier element

8. Optical element

9. Ferrule

10. Insertion slot

11. Heating element

12. Light source

13. Y-fiber

14. Optical fiber connector

15. Detector

16. Excitation arm

17. Beam splitter

18. Focusing/Beam collimating element

19. High pass filter element

20. Lens

21. Analyzing unit

22. Light source

23. Beam splitter

24. Focusing/Beam collimating element

25. Optical element

26. Carrier element

27. Filter element

28. Lens

29. Detector

30. Analyzing device

31. Analyzing unit

32. Light source

33. Beam splitter

34. Focusing/Beam collimating element

35. Carrier element

36. Optical element

37. Filter element

38. Lens

39. Detector

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. An in-vitro method for detecting a target nucleic acid in a sample, comprising:
a. contacting the sample with a probe wherein the probe is labeled with a fluorescent dye and adapted to hybridize with the target nucleic acid;
b. irradiating the probe with an excitation light to excite the fluorescent dye,
c. observing, with a detector, for an emission of light from the fluorescent dye; and
d. determining if the target nucleic acid is in the sample based on a detection of light emitted by the fluorescent dye,
wherein the probe is immobilized to a carrier element prior to contact with the sample, and an optical element is positioned proximate to the carrier element and adapted to permit light emitted from fluorescent dye to pass therethrough to the detector.

2. The method of claim 1, wherein the carrier element is a polymer.

3. The method of claim 2, wherein the carrier element contains at least 20 nmol/1 of the probe.

4. The method of claim 3, wherein the carrier element contains at least 2.5 nmol/1 of the probe.

5. The method of claim 1, wherein the excitation light is a laser beam.

6. The method of claim 1, further comprising, prior to performing step (b), heating the carrier element to at least 65° C., exposing the carrier element to ultraviolet radiation, or a combination of both.

7. The method of claim 1, further comprising defining a peak wavelength of light that is specific to a hybrid of the target nucleic acid and the probe, and concluding that the target nucleic acid is in the sample if the light emitted by the probe is determined to have the defined peak wavelength.

8. An analyzing unit for in-vitro detection of a target nucleic acid, comprising:
a light source configured to emit an excitation light to a carrier element of a sampling unit, and
a detector configured to receive light emitted from a carrier element of a sampling unit; and
a microprocessor configured to receive data from the detector, and to determine if the target nucleic acid is present at carrier element of a sampling unit.

9. The analyzing unit according to the claim 8, further comprising
a beam splitter with a high pass optical filter, or a dichroic mirror configured to eliminate the excitation light from a light that is to be measured by the detector.

10. The analyzing unit according to claim 8, wherein the detector is configured to receive light through an optical element is in the form of an optical fiber or a glass window.

11. The analyzing unit according to claim 8, further comprising at least one focusing element configured to deliver excitation light from the light source to a carrier element of a sampling unit.

12. The analyzing unit according to claim 8, further comprising at least one beam collimating element configured to collect light emitted from a carrier element of a sampling unit.

13. An analyzing device, comprising:
the analyzing unit according to claim 8; and
a sampling unit comprising:
a container for collecting and isolating a sample;
a carrier element for carrying an molecular beacon probe; and
an optical element proximate to the carrier element and adapted for the passage of light to and from the carrier element,
wherein the analyzing unit and the sampling unit are configured to cooperate for the passage of light from the light source of the analyzing unit to the carrier element of the sampling unit, and for the passage of light from the carrier element of the sampling unit to the detector of the analyzing unit, with the analyzing unit further configured for determining if a target nucleic acid is present at the carrier element of the sampling unit.

14. A sampling unit for collecting a sample for in-vitro detection of a target nucleic acid, comprising:
a container comprising an extraction nozzle and a reaction chamber for the collection and isolation of the sample;
a carrier element inside the reaction chamber, the carrier element adapted to carry a wherein the probe labeled with a fluorescent dye; and
Response to Notice to File Missing Parts and Preliminary Amendment an optical element positioned proximate to the carrier element and adapted for the passage of an excitation light into the reaction chamber and to the carrier element, and for the passage of a light emission from the fluorescent dye and out of the reaction chamber.

15. The sampling unit according to claim 14, wherein the carrier element is a polymer.

16. The sampling unit according to claim 15, wherein the polymer is a liquid swelling polymer chosen from: an agarose gel, a hydrogel, a polyamide gel, and a polyester gel.

17. The sampling unit according to claim 14, wherein the carrier element contains at least 20 nmol/1 of the probe.

18. The sampling unit according to claim 17, wherein the carrier element contains at least 2.5 μmol/1 of the probe.

19. The sampling unit according to claim 14, further comprising a ferrule proximate the optical element, the ferrule being adapted for connecting the sampling unit to an analyzing unit with an alignment for the passage of light into and out from the reaction chamber through the optical element.

20. The method of claim 1, wherein the probe is a molecular beacon.

21. The sampling unit of claim 14, wherein the probe is a molecular beacon.


SEQUENCE LISTING (1 sequence)

SEQ ID NO: 1 - DNA, 26 bp, Artificial Sequence
  Corona E-Beacon
acactagccatccttactgcgcttcg